(19) United States
(12) Patent Application Publication
(10) Pub. No.: US 2006/0177501 A1
(43) Pub. Date: Aug. 10, 2006

(54) Film Coated Tablets Containing Ibuprofen

(75) Inventors: Sergej Anschuetz, Ruemmelsheim (DE); Roland Schneider, Sprednlingen (DE); Gerd Jung, Breitenguessbach (DE); Albert Schaupp, Strullendorf (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim International GmbH, Ingelheim (DE)

(21) Appl. No.: 11/275,735

(22) Filed: Jan. 26, 2006

(30) Foreign Application Priority Data
Feb. 9, 2005 (EP) EP 05 002 682

(51) Int. Cl. A61K 9/20; A61K 31/205; A61K 31/192

(52) U.S. Cl. 424/464; 514/570; 514/554

(57) ABSTRACT

The invention relates to film-coated tablets containing ibuprofen lysinate with improved stability, bioavailability and higher acceptance (compliance) in patients, and the use of these film-coated tablets for preparing a pharmaceutical composition for the treatment of acute and/or chronic pain.

### BACKGROUND TO THE INVENTION

1. Technical Field

The invention relates to film-coated tablets containing ibuprofen lysinate with improved stability, bioavailability and higher acceptance (compliance) in patients, and the use of these film-coated tablets for preparing a pharmaceutical composition for the treatment of acute and/or chronic pain.

2. Prior Art

British Patent Application GB 1 471 910 describes ibuprofen lysinate as a water-soluble form of ibuprofen with an improved therapeutic activity. European Patent Application EP 0 172 014 proposes ibuprofen formulations with a high content of ibuprofen, which contain as carrier 1 to 15 wt. % croscarmellose sodium. European Patent Application EP 0 411 952 describes chewable tablets with a rapid release of active substance, wherein the active substance, particularly ibuprofen, is present in the form of compressed granules which have been granulated with polyvinylpyrrolidone and sodium laurylsulphate. European Patent Application EP 0 505 180 proposes a disintegrant-free tablet containing an amount of ibuprofen lysinate of more than 90 wt. %.

The known preparation Dolormin® is a film-coated tablet consisting of ibuprofen lysinate, microcrystalline cellulose, polyvinylpyrrolidone (povidone), magnesium stearate, titanium dioxide, hypromellose and hydroxypropylcellulose.

Polyvinylpyrrolidone (povidone) acts as a dry binder. However, this substance is slightly hygroscopic and causes the corresponding tablet mixtures to have a tendency to stick during compression, particularly at higher relative humidity levels, which may lead to serious disadvantages in the production process (e.g. sticking of the tools). In addition, depending on the total composition of the product, povidone may interact with other substances, e.g. colour changes may occur.

When larger amounts are used in tablet formulations, because of the good water-solubility of the substance, depending on the total composition, a delaying effect on the decomposition time of the preparation cannot be ruled out.

The problem of the present invention was to provide an ibuprofen-containing film-coated tablet the therapeutic activity of which is comparable to or better than that of Dolormin®, while avoiding the disadvantages mentioned above.

### BRIEF DESCRIPTION OF THE INVENTION

It has now been found, surprisingly, that when a particular combination of carriers is used in the tablet core, the use of the dry binder povidone can be dispensed with and the associated disadvantages can be avoided, while at the same time the favourable therapeutic qualities are retained or even improved.

The invention thus relates to an ibuprofen-containing film-coated tablet, wherein the tablet core consists of the following components:

| | | |
|---|---|---|
| (a) | 80.0-85.0 | wt. % of the lysinate of racemic (R/S)-ibuprofen, |
| (b) | 12.5-17.5 | wt. % microcrystalline cellulose, |
| (c) | 0.1-1.0 | wt. % highly dispersed SiO₂, and |
| (d) | 0.75-1.5 | wt. % magnesium stearate. |

The invention further relates to the use of this ibuprofen-containing film-coated tablet for preparing a pharmaceutical composition for treating acute and/or chronic pain.

### DETAILED DESCRIPTION OF THE INVENTION

The term ibuprofen lysinate hereinbefore and hereinafter denotes (±)-2-(p-isobutylphenyl) propionic acid-lysine salt, particularly in the form of the monohydrate.

Preferably, the tablet core consists of (percentages given are based on the tablet core):
(a) 83.0-84.0, particularly about 83.4 wt. % of the lysinate of racemic (R/S)-ibuprofen,
(b) 14.5-15.5, particularly about 15.0 wt. % of microcrystalline cellulose,
(c) 0.3-0.4, particularly about 0.37 wt. % of highly dispersed SiO₂, und
(d) 1.0-1.4, particularly about 1.22 wt. % of magnesium stearate.

Preferably a single film-coated tablet contains 342, 684 or 1026 mg of ibuprofen lysinate, corresponding to 200, 400 or 600 mg of ibuprofen, particularly 684 mg of ibuprofen lysinate, corresponding to 400 mg of ibuprofen.

The microcrystalline cellulose used is preferably Sanaq® 102 or Vivapur® 102, a microcrystalline cellulose with an average polymerisation level of 215-240 and a density (bulk) of 0.28-0.33 g/cm³, which may be obtained for example from JRS—J. Rettenmaier USA LP, Schoolcraft, Mich. 49087.

The highly dispersed silicon dioxide used is preferably Aerosil® 200, a highly pure colloidal silicic acid which may be obtained for example from Degussa AG, Weiβfrauenstrasse 9, 60311 Frankfurt am Main.

The nature of the film-forming agent to be used is non-critical per se. As a rule, an aqueous solution of a film-forming system is applied to the tablet cores. Preferably, one of the materials listed in the following Table 1 is used as the film-forming agent:

TABLE 1

| Brand name | ingredients | Manufacturer |
|---|---|---|
| Opadry ® I | HPMC, PEG & pigment | Colorcon, West Point, PA |
| Opadry II ® | HPMC, PEG, | Colorcon, West Point, PA |
| | maltodextrin & pigment | |
| Klucel ® | hydroxypropylcellulose | Hercules/Aqualon, Wilmington, DE |
| Natrosol ® | hydroxyethylcellulose | Hercules/Aqualon, Wilmington, DE |
| Kollidon ® | polyvinyl pyrrolidone | BASF, Parsippany, NJ |
| Kelton ® | sodium alginate | Kelco, San Diego, CA |
| Pharmaceutical gelatine | gelatine | Hormel Foods Corp., Austin, MN |

| | |
|---|---|
| HPMC: | Hydroxypropylmethylcellulose |
| PEG: | Polyethyleneglycol |
| Pigment: | Titanium dioxide |

Another preferred embodiment of the film-coated tablet according to the invention is an ibuprofen-containing film-coated tablet, the tablet film being produced from Opadry II® made by Colorcon.

Particularly preferred is an ibuprofen-containing film-coated tablet, wherein the tablet film contains 0.5 to 5.0, particularly 2.0 to 3.0 wt. % Opadry II®, based on the total weight of the film-coated tablet.

The tablet according to the invention can be produced by direct mixing and compression of the ingredients or by granulation and compression.

The film solution is prepared by mixing the film-forming agent with water. This solution can be applied to the tablet cores using a conventional coating pan.

The compression forces required to produce tablets with suitable breaking strength and hence the desired breakdown times are dependent on the shapes and sizes of the punching tools used. Preferably, the compression force is in the range from 2-20 kN. Higher compression forces may lead to tablets with a slower release of active substance. Lower compression forces may lead to mechanically unstable tablets. The tablet cores may take different forms; round biplanar or biconvex and oval or oblong shapes are preferred.

The film-coated tablets according to the invention are suitable for the treatment of acute and chronic pain, particularly headaches, toothache and menstrual pain and migraine. With the film-coated tablet according to the invention high concentrations sufficient to combat the pain are achieved very rapidly in the blood plasma and activity site.

The following Example serves to illustrate formulations according to the invention. It is intended merely as a possible method described by way of example, without restricting the invention to its content.

### EXAMPLE 1

| composition | per tablet mg | brand name or supplier |
|---|---|---|
| ibuprofen-lysinate | 684.000 | |
| microcrystalline cellulose 102 | 123.000 | Sanaq ® 102/Vivapur ® |
| | | 102 |
| highly dispersed silicon dioxide | 3.000 | Aerosil ® 200 |
| magnesium stearate | 10.000 | |
| tablet core | 820.000 | |
| Opadry ® white + | 20.000 | Colorcon |
| 6% manufacturer's additives | 1.200 | |
| purified water* | 152.000 | |
| | 840.000 | |

*not present in the finished product

The direct compression comprises preparing a mixture of the ingredients of the tablet core with a mixer. The mixture is screened and compressed to form tablets with rounded edges.

Then a solution of the film-forming agent in water is prepared, which is applied to the tablets.

In tests carried out in vitro the tablets according to the invention thus obtained demonstrate release characteristics for the active substance which are comparable with or in some cases slightly faster and more uniform than those of Dolormin®.

1. An Ibuprofen-containing film-coated tablet, wherein the tablet core consists of the following components:

| | | |
|---|---|---|
| (a) | 80.0-85.0 | wt. % of the lysinate of racemic (R/S)-ibuprofen, |
| (b) | 12.5-17.5 | wt. % of microcrystalline cellulose, |
| (c) | 0.1-1.0 | wt. % of highly dispersed SiO₂, and |
| (d) | 0.75-1.5 | wt. % of magnesium stearate, |

the percentages given for the respective ingredients being based on the total weight of the film-coated tablet.

2. The Ibuprofen-containing film-coated tablet according to claim 1, wherein the tablet core consists of the following components:

| | | |
|---|---|---|
| (a) | 83.0-84.0 | wt. % of the lysinate of racemic (R/S)-ibuprofen, |
| (b) | 14.5-15.5 | wt. % of microcrystalline cellulose, |
| (c) | 0.3-0.4 | wt. % of highly dispersed SiO₂, and |
| (d) | 1.0-1.4 | wt. % of magnesium stearate. |

3. The Ibuprofen-containing film-coated tablet according to claim 1, wherein the tablet film has been produced from Opadry II®.

4. The Ibuprofen-containing film-coated tablet according to claim 1, wherein the amount of the tablet film is 0.5 to 5.0 wt. % based on the total weight of the film-coated tablet.

5. The Ibuprofen-containing film-coated tablet according to claim 4, wherein the amount of the tablet film is 2.0 to 3.0 wt. % based on the total weight of the film-coated tablet.

6. A method of treating acute and/or chronic pain comprising administering to a patient in need thereof the Ibuprofen-containing film-coated tablet according to claim 1.

7. A method for rapidly achieving a high enough blood plasma and activity site concentration to combat pain comprising administering to a patient in need thereof the Ibuprofen-containing film-coated tablet according to claim 1.

8. The Ibuprofen-containing film-coated tablet according to claim 2, wherein the tablet film has been produced from Opadry II®.

9. The Ibuprofen-containing film-coated tablet according to claim 2, wherein the amount of the tablet film is 0.5 to 5.0 wt. % based on the total weight of the film-coated tablet.

10. The Ibuprofen-containing film-coated tablet according to claim 3, wherein the amount of the tablet film is 0.5 to 5.0 wt. % based on the total weight of the film-coated tablet.

11. The Ibuprofen-containing film-coated tablet according to claim 8, wherein the amount of the tablet film is 0.5 to 5.0 wt. % based on the total weight of the film-coated tablet.

12. The Ibuprofen-containing film-coated tablet according to claim 9, wherein the amount of the tablet film is 2.0 to 3.0 wt. % based on the total weight of the film-coated tablet.

13. The Ibuprofen-containing film-coated tablet according to claim 10, wherein the amount of the tablet film is 2.0 to 3.0 wt. % based on the total weight of the film-coated tablet.

14. The Ibuprofen-containing film-coated tablet according to claim 11, wherein the amount of the tablet film is 2.0 to 3.0 wt. % based on the total weight of the film-coated tablet.
